(19) United States
(12) Patent Application Publication
(10) Pub. No.: US 2022/0072229 A1
(43) Pub. Date: Mar. 10, 2022

(54) ELECTRONIC WEARABLE PATCH FOR MEDICAL USES

(71) Applicant: OCELLA INC., Indianapolis, IN (US)

(72) Inventor: Rajan KUMAR, Bloomington, IN (US)

(21) Appl. No.: 17/309,957

(22) PCT Filed: Jan. 14, 2020
(86) PCT No.: PCT/US2020/013582

§ 371 Date: Jul. 5, 2021

Related U.S. Application Data
(60) Provisional application No. 62/792,017, filed on Jan. 14, 2019.

(51) Int. Cl. A61M 5/172; A61K 9/70; A61B 5/00; A61B 5/024; A61B 5/01; G16H 20/17

(52) U.S. Cl. CPC A61M 5/1723; A61K 9/7092; A61B 5/6833; A61B 5/02438; A61B 5/01; A61B 5/0022; A61M 2205/0216; A61B 5/0006; A61B 5/0008; A61B 2562/164; A61B 2562/166; A61M 2205/8206; A61M 2205/3584; G16H 20/17

(57) ABSTRACT

An electronic wearable patch for medical uses is an apparatus for medical monitoring, medication management or patient compliance, wellness management, prevention, or other medical uses. The apparatus includes a controller, at least one sensing module, a wireless communication module, a flexible energy-storage module, a flexible printed circuit board (PCB), and a flexible adhesive. The controller manages and controls the at least one sensing module and the wireless communication module. The at least one sensing module collects raw data. The wireless communication module sends the raw data to an external computing device, which processes the raw data into useable medical data. The flexible energy-storage module electrically powers the electronic componentry of the apparatus. The flexible PCB allows for the electronic connections between the controller, the at least one sensing module, and the wireless communication module. The flexible adhesive attaches the apparatus to a user's skin.

This application is a national phase application of PCT/US2020/013582, filed Jan. 14, 2020, which claims priority to U.S. Provisional Patent Application No. 62/792,017, filed on Jan. 14, 2019. Each of the above-listed applications is incorporated by reference herein in its entirety.

### FIELD OF THE INVENTION

The present disclosure generally relates to medical equipment. More specifically, the present disclosure describes flexible electronic apparatuses to facilitate medical monitoring and medication management.

### BACKGROUND OF THE INVENTION

Flexible electronic devices include assembling electronic circuits by mounting electronic devices on flexible substrate, such as those made of plastic. Flexible circuits may also include printing of the electronic components on the flexible substrates.

Flexible electronic devices may be used as connectors in applications where traditional hard board circuits, or hard wiring may not be used due to a limit of available space, or due to flexibility constraints.

However, existing thermal processes lead to a slow manufacturing of flexible electronic devices. Further, a high-pulse current is required for Bluetooth

communications which may be beyond the capabilities of current flexible electronic devices. Further, existing flexible electronic devices may require external sources of energy and may not include inbuilt flexible batteries. Further, existing flexible electronic devices may not be used for medical purposes, including medication management, and medical monitoring.

Therefore, there is a need for improved flexible electronic apparatuses to facilitate medical monitoring and medication management that may overcome one or more of the above-mentioned problems and/or limitations.

### SUMMARY OF THE INVENTION

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

According to some embodiments, a wearable patch that may be applied to the skin for medical monitoring, medication management or patient compliance, wellness management, and prevention is disclosed. The wearable patch may comprise of a flexible or stretchable circuit on a flexible or stretchable substrate with electronic components, custom-shaped, and supercapacitor, and wireless communication.

Further, the wearable patch may include one or more sensors, such as potentiostats, accelerators, gyroscopes, and so on that may interface with the skin of a user on the surface or underneath the surface of the skin, such as with an implantable needle or microneedle. Further, the one or more sensors may be configured to measure a plurality of physiological parameters corresponding to the user, including heart rate, blood pressure, body temperature,

Further, the wearable patch may include a processing device and one or more logic chips to analyze the plurality of physiological parameters corresponding to the user and determine one or more medical conditions.

Further, the wearable patch may include a flexible battery integrated with a UV curable ink.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants.

In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is a schematic view illustrating a preferred embodiment of the present invention.

FIG. 2 is a block diagram illustrating the electronic connections in the present invention.

FIG. 3 is a block diagram illustrating the electrical connections in the present invention.

FIG. 4 is a schematic view illustrating an exemplary embodiment of a flexible energy-storage module for the present invention.

FIG. 5 is an illustration of an online platform consistent with various

embodiments of the present disclosure.

FIG. 6 shows a wearable patch that may be applied to the skin for medical monitoring, medication management or patient compliance, wellness management, and prevention, in accordance with some embodiments. FIG. 7 shows an exemplary flowchart of a method of manufacturing a flexible electronic device and a wearable patch including the flexible electronic device, in accordance with some embodiments.

FIG. 8 shows an exemplary flowchart of a method of manufacturing a flexible electronic device and a wearable patch including the flexible electronic device, in accordance with some embodiments.

FIG. 9 shows an exemplary assembly line to facilitate manufacturing a flexible electronic device and a wearable patch including the flexible electronic device, in accordance with some embodiments.

FIG. 10 shows an exemplary method of manufacturing a flexible electronic device and a wearable patch including the flexible electronic device, in accordance with some embodiments.

FIG. 11 shows an exemplary representation of a flexible electronic device, displaying a plurality of layers of the flexible electronic device, in accordance with some embodiments.

FIG. 12 is a block diagram of a computing device for implementing the methods disclosed herein, in accordance with some embodiments.

### DETAILED DESCRIPTION OF THE INVENTION

All illustrations of the drawings are for the purpose of describing selected versions of the present invention and are not intended to limit the scope of the present invention.

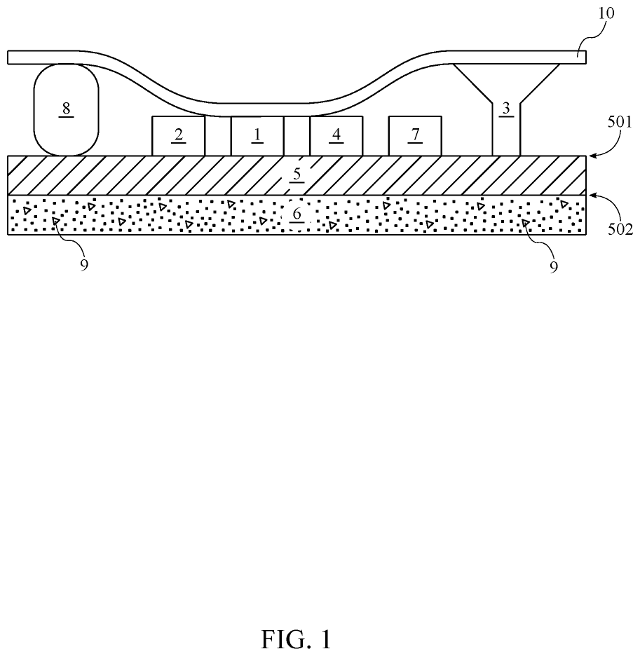

The present invention is an electronic wearable patch for medical uses including, but not limited to, medical monitoring, medication management or patient compliance, wellness management, and prevention. The componentry of the present invention is flexible or stretchable so that a user can comfortably wear the present invention. As can be seen in FIGS. 1 through 3, a preferred embodiment of the present invention comprises a controller 1, at least one sensing module 2, a wireless communication module 3, a flexible energy-storage module 4, a flexible printed circuit board (PCB) 5, and a flexible adhesive 6. The controller 1 is used to manage the functionalities of the other electronic components of the present invention. The at least one sensing module 2 allows the present invention to collect the raw data that can be eventually processed into usable medical data for the user. Moreover, the wireless communication module 3 allows the present invention to send the raw data to an external computing device, such as a mobile computing device (e.g., a smart-phone, smart-watch, a piece of smart-clothing, and a smart-tag), a laptop, a desktop, or a tablet personal computer, which is better at processing the raw data into the usable medical data for the user. The external computing device is then able to relay notifications or the useable medical data to medical professionals or personal contacts that are associated to the user of the present invention. The wireless communication module 3 can be, but is not limited to, a personal area network module (e.g., Bluetooth), a local area network module (e.g., WiFi), or a near field communication module (e.g., radio frequency identification [RFID]). The flexible energy-storage module 4 is used to electrically power the other electronic components of the present invention and can be flexed without compromising its structural integrity or its functionality. The flexible PCB 5 provides a base for the other electronic components to be mounted onto the present invention and can also be flexed without compromising its structural integrity or its functionality. The flexible adhesive 6 is used to stick the present invention on the user's skin and can similarly be flexed without compromising its structural integrity or its functionality.

The general configuration of the aforementioned components allows the present invention to efficiently and effectively provide electronic functionalities for medical uses without a cumbersome rigid structure. The flexible PCB 5 comprises a first board face 501 and a second board face 502. The first board face 501 is typically oriented away from the user's skin, while the second board face 502 is typically oriented towards the user's skin. Thus, the controller 1, the at least one sensing module 2, the wireless communication module 3, and the flexible energy-storage module 4 are mounted onto the first board face 501, and the flexible adhesive 6 are mounted across the second board face 502. This arrangement prevents the controller 1, the at least one sensing module 2, the wireless communication module 3, and the flexible energy-storage module 4 from interfering with the adhesion of the present invention to the user's skin by the flexible adhesive 6. Moreover, the controller 1, the at least one sensing module 2, and the wireless communication module 3 are electronically connected to each other by the flexible PCB 5, which allows the controller 1, the at least one sensing module 2, and the wireless communication module 3 to communicate amongst each other. In addition, the controller 1, the at least one sensing module 2, and the wireless communication module 3 are electrically connected to the flexible energy-storage module 4 so that the flexible energy-storage module 4 is able to readily power the controller 1, the at least one sensing module 2, and the wireless communication module 3.

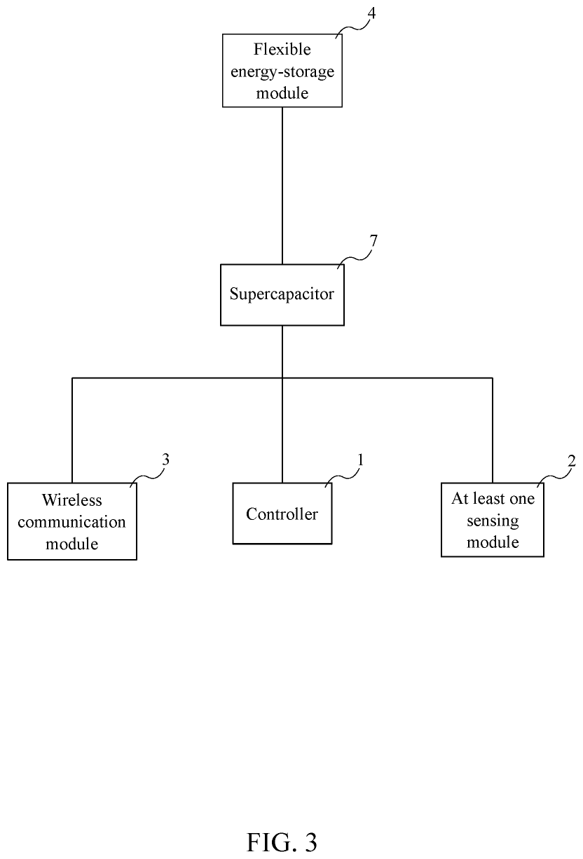

As can be seen in FIG. 3, the present invention may further comprise a supercapacitor 7, which is used to act an electrical intermediary between the flexible energy-storage module 4 and the electronic components of the present invention. The supercapacitor 7 is mounted onto the first board face 501 in order to firmly secure the supercapacitor 7 to the present invention. Moreover, the controller 1, the at least one sensing module 2, and the wireless communication module 3 are electrically connected to the flexible energy-storage module 4 through the supercapacitor 7, which allows the flexible energy-storage module 4 to initially charge the supercapacitor 7 and then allows the supercapacitor 7 to readily deliver electrical power to the controller 1, the at least one sensing module 2, and the wireless communication module 3.

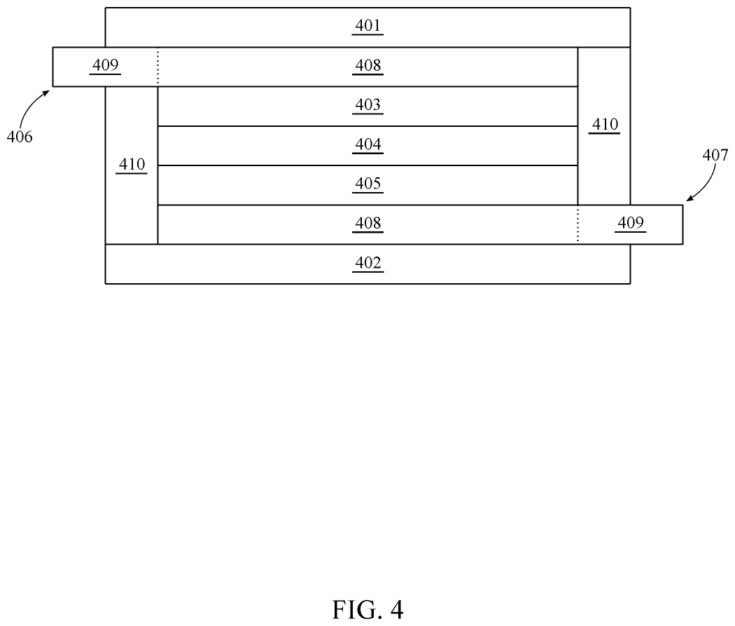

The flexible energy-storage module 4 is a generally flat layered body. Thus, as can be seen in FIG. 4, the flexible energy-storage module 4 may comprise a first substrate layer 401, a second substrate layer 402, a cathode layer 403, an electrolytic separation layer 404, an anode layer 405, a negative connection layer 406, a positive connection layer 407, and a sealing adhesive 410. The first substrate layer 401 and the second substrate layer 402 are base layers that enclose the other layers of the flexible energy-storage module 4. The cathode layer 403, the electrolytic separation layer 404, and the anode layer 405 function together as an electrolytic battery cell. The negative connection layer 406 and the positive connection layer 407 function as the terminals of the aforementioned electrolytic battery cell with their respective electrical charges. In addition, the first substrate layer 401, the negative connection layer 406, the cathode layer 403, the electrolytic separation layer 404, the anode layer 405, the positive connection layer 407, and the second substrate layer 402 are serially superimposed onto each other. The serial order for these layers allows for the necessary electrochemistry to readily provide electrical power to the electronic components of the present invention. Moreover, the first substrate layer 401 and the second substrate layer 402 are peripherally fixed to each other by the sealing adhesive 410, which encloses the electrochemical process implemented by the other layers of the flexible energy-storage module 4.

The negative connection layer 406 and the positive connection layer 407 need to be able to electrically connect to the electronic components outside of the enclosure formed by the first substrate layer 401, the second substrate layer 402, and the sealing adhesive 410. Thus, the negative connection layer 406 and the positive connection layer 407 may each further comprise a base portion 408 and a tab portion 409, which are also shown in FIG. 4. The base portion 408 is used to receive the electrical charge from their respective electrodes and is confined to the enclosure formed by the first substrate layer 401, the second substrate layer 402, and the sealing adhesive 410. Consequently, the sealing adhesive 410 is positioned around the base portion 408 of the negative connection layer 406, the cathode layer 403, the electrolytic separation layer 404, the anode layer 405, and the base portion 408 of the negative connection layer 406. Moreover, the tab portion 409 is peripherally positioned to the base portion 408, which allows the tab portion 409 of the negative connection layer 406 to traverse through the sealing adhesive 410 and allows the tab portion 409 of the positive connection layer to similarly traverse through the sealing adhesive 410. Consequently, the tab portion 409 is able to electrically connect to the electronic components outside of the enclosure formed by the first substrate layer 401, the second substrate layer 402, and the sealing adhesive 410.

The manufacturing method for the flexible energy-storage module 4 is preferably through a coating method (e.g., screen-printing, spray-coating, lamination, and inkjet printing) and preferably use an ultraviolet (UV) curable ink in order to form the necessary layers in the coating method. Thus, the cathode layer 403 and the anode layer 405 are preferably a pair of printable coatings, which is made of an ultraviolet (UV) curable polymer. The UV curable polymer allows the cathode layer 403 and the anode layer 405 to quickly dry in the manufacturing process and to maintain their structural flexibility. More specifically, the UV curable polymer is made of at least one material constituent selected from a group consisting of: at least one monomer, at least one oligomer, at least one dispersant, at least one self-level additive, at least one conductive-particle additive, and combinations thereof. The at least one dispersant is used to improve the distribution of the material constituent in the UV curable polymer. The at least one self-level additive ensures the UV curable polymer to form a leveled interface between the ink and air. The at least one conductive-particle additive is used to improve the conductivity of the UV curable polymer. In addition, the electrolytic separation layer 404 is preferably a UV curable gel-like structure that may be another printable coating. Similar to the UV curable polymer, the UV curable gel-like structure allows the electrolytic separation layer 404 to quickly dry in the manufacturing process and to maintain its structural flexibility. More specifically, the UV curable gel-like structure is made of at least one material constituent selected from a group consisting of: at least one dissolved salt (e.g., LiOH or KOH), at least one salt additive, and combinations thereof.

A specific embodiment resulting from the manufacturing method for the flexible energy-storage module 4 dimensions a combined thickness of the first substrate layer 401 and the negative connection layer 406 at 50 microns. This specific embodiment further dimensions the cathode layer 403 at 100 microns. In addition, this specific embodiment similarly dimensions combined thickness of the second substrate layer 402 and the positive connection layer at 50 microns. This specific embodiment also dimensions the anode layer 405 at 200 microns.

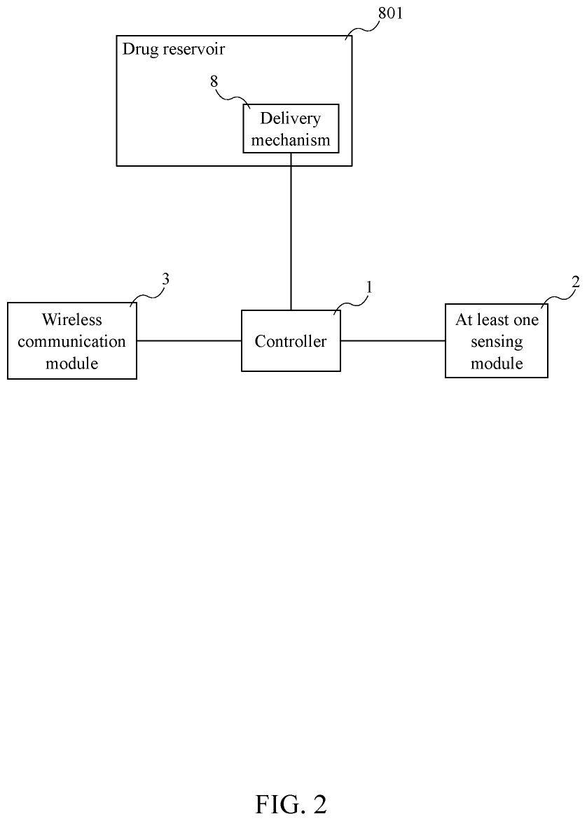

As can be seen in FIGS. 1 and 2, the present invention may further comprise a drug reservoir 8, which is used to retain and readily dispense medicine to the user. The medicine retained by the drug reservoir 8 can be for, but is not limited to, a medical use, a cosmetic use, or a wellness use (e.g., vitamins, minerals, and so on). The drug reservoir 8 is mounted onto the first board face 501 in order to firmly secure the drug reservoir 8 to the present invention. Moreover, the drug reservoir 8 comprises a delivery mechanism 801 that allows the medicine to travel from the drug reservoir 8 into the user. The delivery mechanism 801 is electronically connected with the controller 1 so that the controller 1 is able to readily actuate the delivery mechanism 801 and consequently dispense the medicine to the user. The delivery mechanism 801 can be, but is not limited to, diffusion of the medicine through the user's skin or a set of microneedles that are actuated by an electronic connection to the controller 1. As can be seen in FIG. 1, the present invention may further comprise a quantity of wound-treating compound 9, which allows the present invention to medicate a wound in the user's skin if the present invention is applied over the wound. The quantity of wound treating compound 9 can be, but is limited to, an antiseptic, a soothing ointment, or combinations thereof. In addition, the quantity of wound-treating compound 9 is integrated into the flexible adhesive 6 so that the quantity of wound-treating compound 9 is in tactile contact with the user's skin through the flexible adhesive 6 and is able to discharge into the user's skin.

As can be seen in FIG. 1, the present invention may further comprise a color coating 10, which is configured to be the same color as the user's skin and prevents the present invention from being visually noticeable. Moreover, the color coating 10 is mounted across the first board face 501, which allows the color coating 10 to camouflage the entirety of the present invention against the user's skin.

The at least one sensing module 2 is able to interface with the user's skin either on the skin surface or underneath the skin surface (e.g., with an implantable needle or microneedle). The at least one sensing module 2 is preferably selected from the group consisting of: at least one potentiostat sensor, at least one hydration sensor, at least one temperature sensor, at least one chemical sensor, at least one physical sensor, and combinations thereof. The at least one physical sensor can be used for, but is not limited to, electrocardiogram (EKG or ECG), tracking motion, fall detection, or combinations thereof. The at least one chemical sensor can be used for, but is not limited to, diabetes tracking.

### Supplemental Description

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of flexible electronic apparatuses to facilitate medical monitoring and medication management, embodiments of the present disclosure are not limited to use only in this context.

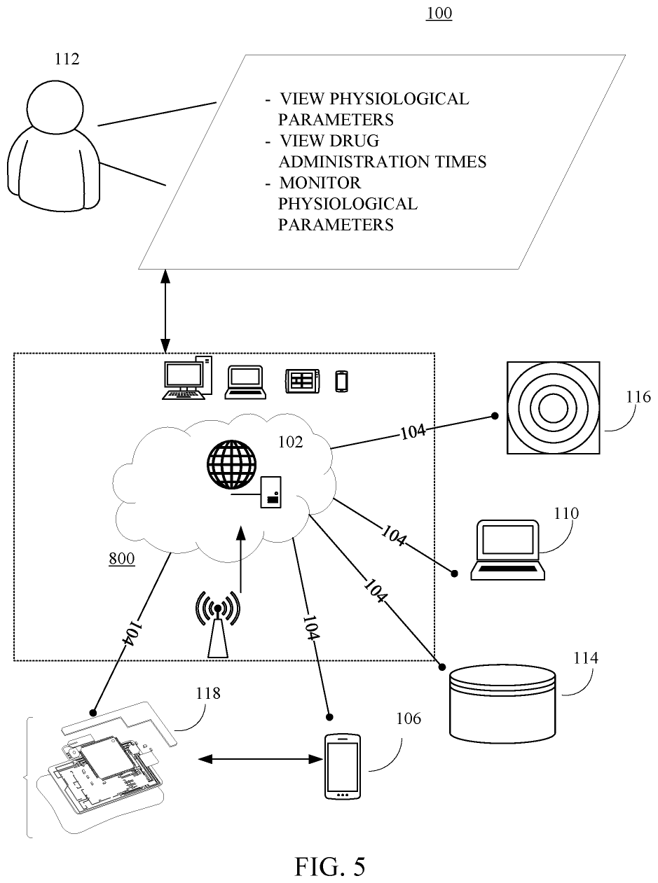

FIG. 5 is an illustration of an online platform 100 consistent with various embodiments of the present disclosure. By way of non-limiting example, the online platform 100 to facilitate medical monitoring and medication management may be hosted on a centralized server 102, such as, for example, a cloud computing service. The centralized server 102 may communicate with other network entities, such as, for example, a mobile device 106 (such as a smartphone, a laptop, a tablet computer etc.), other electronic devices 110 (such as desktop computers, server computers etc.), databases 114, sensors 116, actuators (not shown) and a flexible electronic apparatus 118 over a communication network 104, such as, but not limited to, the Internet. Further, users of the online platform 100 may include relevant parties such as, but not limited to, end users, medical professionals, doctors, and administrators. Accordingly, in some instances, electronic devices operated by the one or more relevant parties may be in communication with the platform. Further, in some embodiments, one or more components including the electronic devices operated by the one or more relevant parties such as the flexible electronic apparatus 118 and the mobile device 106 may be configured to communicate with each other over a communication network, such as a short range communication network including Bluetooth, Wi-Fi, and so on.

A user 112, such as the one or more relevant parties, may access online platform 100 through a web based software application or browser. The web based software application may be embodied as, for example, but not be limited to, a website, a web application, a desktop application, and a mobile application compatible with a computing device 800.

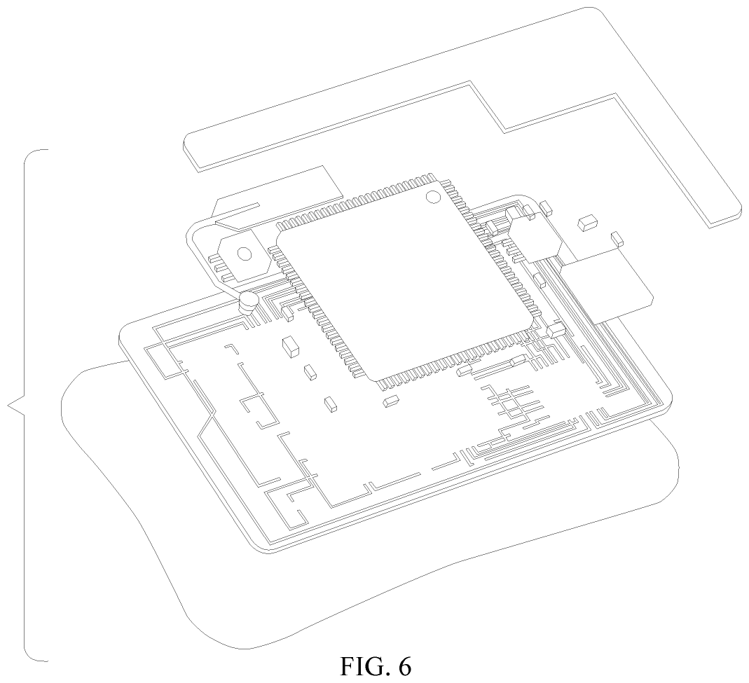

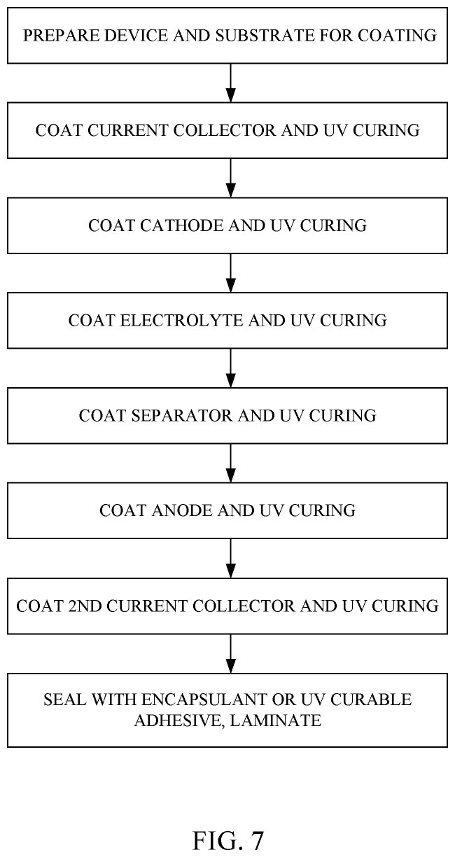

FIG. 6 shows a wearable patch that may be applied to the skin for medical monitoring, medication management or patient compliance, wellness management, and prevention. The wearable patch, in some embodiments, may comprise of a flexible or stretchable circuit on a flexible or stretchable substrate with electronic components, custom-shaped, flexible battery, and supercapacitor, and wireless communication.

Further, the wearable patch may include one or more sensors, such as potentiostats, accelerators, gyroscopes, and so on that may interface with the skin of a user on the surface or underneath the surface of the skin, such as with an implantable needle or microneedle. Further, the one or more sensors may be configured to measure a plurality of physiological parameters corresponding to the user, including heart rate, blood pressure, body temperature.

Further, the wearable patch may include a processing device and one or more logic chips to analyze the plurality of physiological parameters corresponding to the user and determine one or more medical conditions. Accordingly, the wearable patch may be configured for chemical sensing and physical sensing.

The wearable patch may measure the one or more physiological parameters received as inputs from the one or more sensors, and analyze the one or more inputs such as through an algorithm and determine one or more medical conditions.

For instance, the analyzing may include comparing a value of the one or more physiological parameters against pre-set values corresponding to medical symptoms. For e.g., the physiological parameters may include body temperature and heart rate, which may be analyzed to determine fever (high body temperature) and elevated heart rate (higher than a pre-set value). Further, the one or more medical symptoms determined by analyzing the one or more physiological parameters may be used for one or more purposes such as for medical monitoring, medication management, wellness management, and prevention.

Further, the wearable patch may include a communication device, such as a transceiver configured to receive and transmit information, configured to communicate with a plurality of external devices, servers, and so on over a communication network, such as Bluetooth, Wi-Fi, RFID, and so on. For instance, the wearable patch may be configured to communicate with one or more user devices, including smartphones, smartwatches, laptop computers, and so on of a user, and transmit at least one of the one or more physiological parameters and medical conditions. For instance, the wearable patch may be configured to communicate with a smartphone of the user and transmit a blood pressure level of the user, such as above or below a pre-defined range.

Further, in an embodiment, the wearable patch may be configured to facilitate wound healing. The wearable patch may be configured to be applied over a wound and may interface with the wound through an adhesive patch configured to interface with the skin and the wound. Further, the wearable patch may include a wound healing compound, such as a medicine, or an antiseptic, that may be applied to the wound upon application of the wearable patch. Further, in an instance, the wearable patch may include a drug reservoir containing one or more wound healing drugs. Accordingly, based on a severity and condition of the wound, as determined by the wearable patch, the wearable patch may be configured to discharge the one or more wound healing drugs over an adhesive patch interfacing with the wound.

Further, in an embodiment, the wearable patch may be configured to facilitate drug delivery. The wearable patch may include a drug reservoir containing one or more drugs, such as medical drugs, cosmetic drugs, or wellness drugs (including vitamins, minerals, and so on). Further, the wearable patch may be configured to deliver the one or more drugs, such as based on a pre-set timed schedule, or on the basis of one or more medical symptoms determined by the wearable patch. Further, the wearable patch may be configured to transmit a notification upon drug delivery, including describing a drug that may have been delivered, along with a possible medical symptom that may have prompted the drug delivery.

Further, the flexible battery of the wearable patch integrated with a UV curable ink may provide improved manufacturing and reduce a cost of manufacturing compared to other methods of manufacturing.

Further, the flexible or stretchable battery (energy storage device) of the wearable patch may consist of an electric connection, cathode, an anode, a separator between the anode and the cathode and an electrolyte comprising a UV curable polymer. Further, the separator may consist of a polymeric membrane forming a microporous layer. The separator may be chemically and electrochemically stable with regard to the electrolyte and electrode materials and mechanically strong enough to withstand high tension during battery construction. Further, the separator may consist of materials including cellulose, nonwoven fibers (such as polyesters and glass), polymer films (such as polyethylene, and polypropylene), ceramic and naturally occurring substances (such as rubber, or asbestos). Further, in an embodiment, the UV curable polymer may be a gel like electrolyte, a solid electrolyte, a film like electrolyte, and so on, and may be made up of materials including, but not limited to PVdF group polymers, PAN group polymers, PVC group polymers or any combinations of the materials. Further, the flexible battery may include an alkaline battery, a zinc-carbon battery, a lithium and silver oxide battery, a lithium ion battery, a nickel metal hydride (NiMH battery, and so on.

Further, the UV/EB curable ink for the flexible battery may include an electrode active component, secondary electrode active material, additives such as solvent, levelers, stabilizers, and so forth. Further, the UV/EB curable ink for the flexible battery may include an ultra-violet (UV) or electron beam (EB) cured matrix comprising a crosslink reaction product from one or more precursors selected from the group consisting of one or more monomers, one or more oligomers, and combinations thereof. In an embodiment, the flexible battery (energy storage system) may comprise an electrode active material and an ultra-violet (UV) or electron beam (EB) cured matrix comprising a crosslink reaction product from one or more precursors selected from the group consisting of one or more monomers, one or more oligomers, and combinations thereof.

Further, one or more components of the flexible battery may be manufactured by mixing the electrode active material with a curable binder mixture comprising the one or more monomers, one or more oligomers, or a combination thereof to form a slurry, and subjecting the slurry to ultraviolet (UV) or electron beam (EB) radiation, thereby curing the curable binder mixture and forming a UV or EB cured matrix, wherein the UV or EB cured matrix may adhere to ceramic particulate material and the ceramic particulate material may be distributed throughout the UV or EB cured matrix.

Further, the slurry, which may be UV curable, may be coated as repetitive, thin layers into a circuit board to create the wearable patch (flexible electronic device).

Accordingly, the flexible electronic device may comprise UV curable and printable, flexible battery, a microcontroller, a drug reservoir, a communication device, adhesive substrate, color-coating, and a printed circuit board that may be adhered to the skin, where the device may deliver therapeutic drugs through diffusion of the skin or minimally invasive microneedles by communication through smart mobile devices. Further, the flexible electronic device may be adhered to the skin and may monitor physiological parameters related to a user, including blood sugar, hydroencephalitis, hydration, vitals, and so on. Further, the flexible electronic device may include a UV curable and printable flexible battery, a microcontroller, at least one of a chemical and physical sensor, a communication device, an adhesive substrate, color-coating, and a printed circuit board.

FIG. 7 shows an exemplary flowchart of a method of manufacturing a flexible electronic device and a wearable patch including the flexible electronic device. The method may include a step of preparation of device and substrate for coating. Further, the method may include a step of coating current collector and UV curing. Further, the method may include a step of coating cathode and UV curing. Further, the method may include a step of coating electrolyte and UV curing. Further, the method may include a step of coating separator and UV curing. In an embodiment, the separator may not be UV cured, and may be composed of an alternate compound, such as cellulose. Further, the method may include a step of coating anode and UV curing. Further, the method may include a step of coating second current collector and UV curing. Further, the method may include a step of sealing with an encapsulant or UV curable adhesive and laminating.

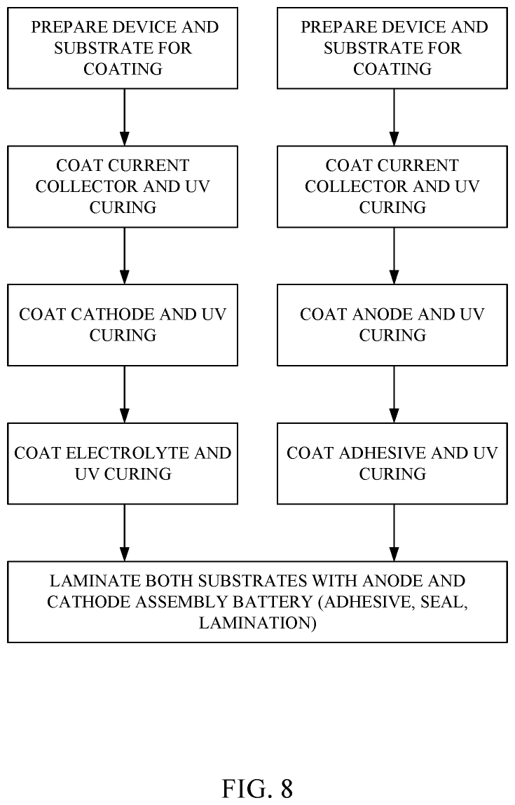

FIG. 8 shows an exemplary flowchart of a method of manufacturing a flexible electronic device and a wearable patch including the flexible electronic device. The method may include a step of preparation of device and substrate for coating on a first substrate. Further, the method may include a step of coating current collector and UV curing the first substrate. Further, the method may include a step of coating cathode and UV curing the first substrate. Further, the method may include a step of coating electrolyte and UV curing the first substrate. Further, the method may include a simultaneous step of preparation of device and substrate for coating on a second substrate. Further, the method may include a step of coating current collector and UV curing the second substrate. Further, the method may include a step of coating anode and UV curing the second substrate. Further, the method may include a step of coating adhesive and UV curing the second substrate. Further, the method may include a step of laminating the first substrate and the second substrate with a cathode and anode assembly battery, and further application of adhesive, a seal, and a final lamination.

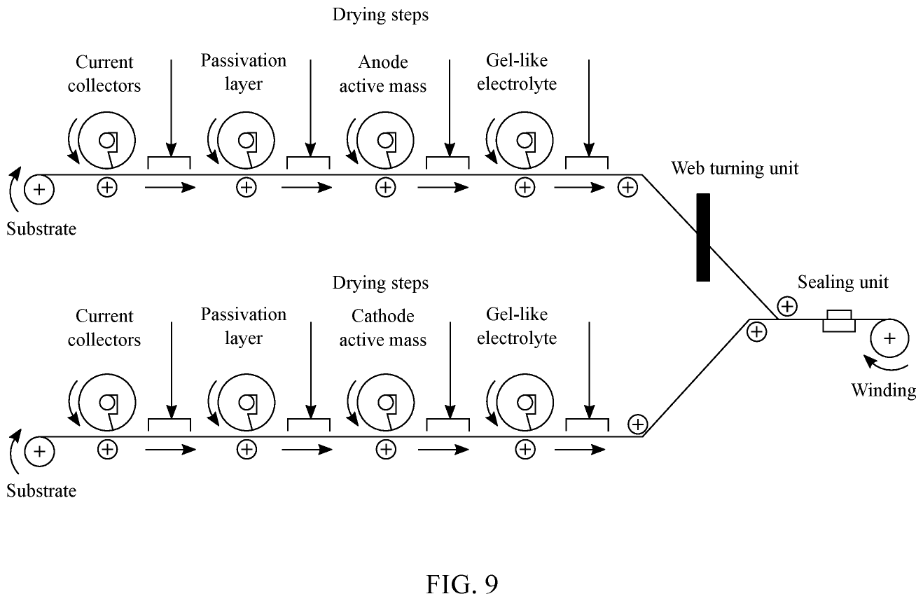

FIG. 9 shows an exemplary assembly line to facilitate manufacturing a flexible electronic device and a wearable patch including the flexible electronic device. The assembly line may include a first lane, and a second lane merging with each other. The first lane may include stations for application of current collectors, a passivation layer, an anode active mass, and an electrolyte on a flexible substrate, along with curing stations. The second lane may include stations for application of current collectors, a passivation layer, a cathode active mass, and a gel-like an electrolyte on a flexible substrate, along with curing stations. Further, the assembly line may include a web turning unit, a sealing unit, and a winding unit. Further, a passivation layer acts a barrier or shield between the internal device and external atmosphere or surrounding area.

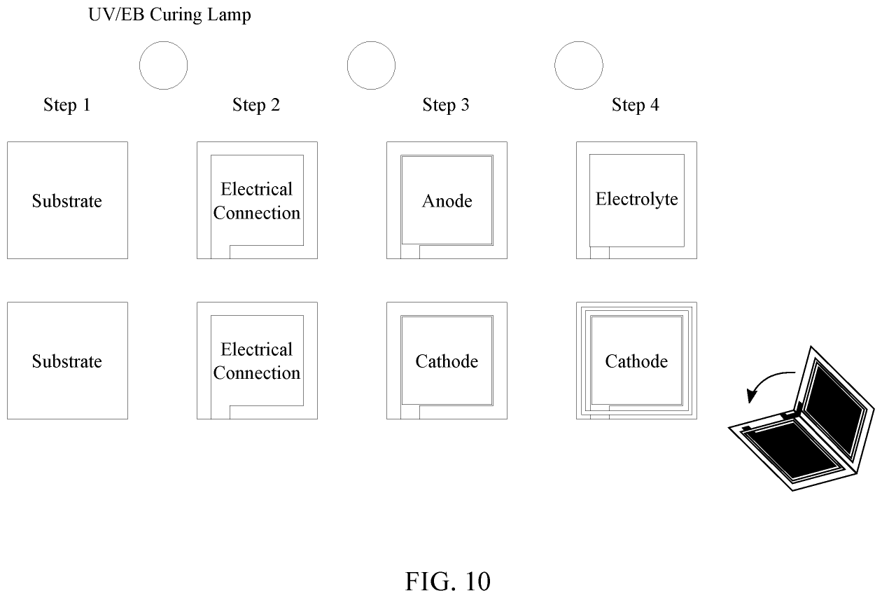

FIG. 10 shows an exemplary method of manufacturing a flexible electronic device and a wearable patch including the flexible electronic device. The method may include a step of preparation of a first substrate and a second substrate. Further, the method may include a step of application of an electric connection on the first substrate and the second substrate. Further, the method may include a step of application of anode and cathode respectively on the first substrate and the second substrate. Further, the method may include a step of application of electrolyte, and cathode and adhesive respectively on the first substrate and the second substrate. Further, the method may include UV curing of the substrate between the plurality of steps. Further, the thickness of the substrate and current collector may be 50 microns after curing, the thickness of the cathode and anode may be 100 microns and 200 microns respectively after curing.

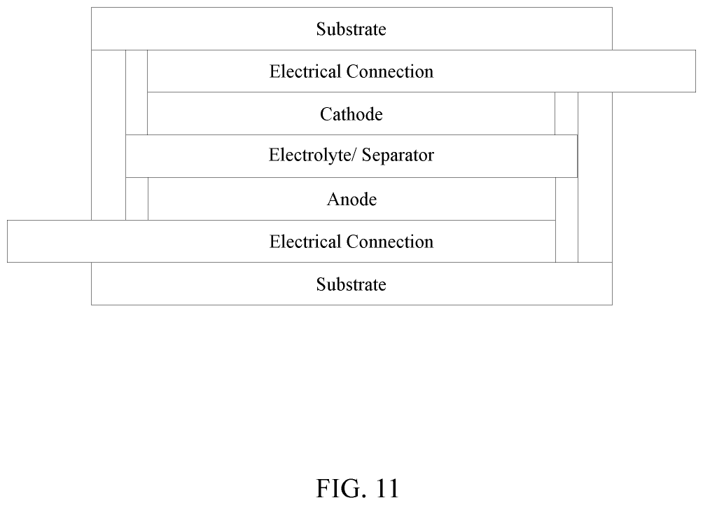

FIG. 11 shows an exemplary representation of a flexible electronic device, displaying a plurality of layers of the flexible electronic device. The flexible electronic device may include a first and a seventh layer of a substrate. Further, the flexible electronic device may include a second and a sixth layer of electrical connections. Further, the flexible electronic device may include a third and a fifth layer of cathode and anode respectively. Further, the flexible electronic device may include a fourth layer of electrolyte and separator.

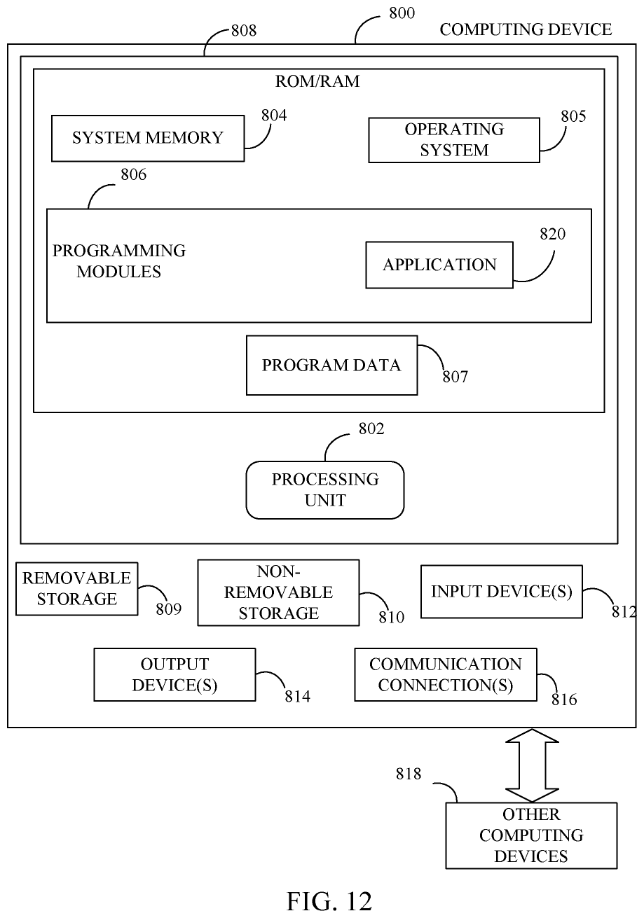

With reference to FIG. 12, a system consistent with an embodiment of the disclosure may include a computing device or cloud service, such as computing device 800. In a basic configuration, computing device 800 may include at least one processing unit 802 and a system memory 804. Depending on the configuration and type of computing device, system memory 804 may comprise, but is not limited to, volatile (e.g., random-access memory (RAM)), non-volatile (e.g., read-only memory (ROM)), flash memory, or any combination. System memory 804 may include operating system 805, one or more programming modules 806, and may include a program data 807. Operating system 805, for example, may be suitable for controlling computing device 800's operation. In one embodiment, programming modules 806 may include machine learning module. Furthermore, embodiments of the disclosure may be practiced in conjunction with a graphics library, other operating systems, or any other application program and is not limited to any particular application or system. This basic configuration is illustrated in FIG. 8 by those components within a dashed line 808.

Computing device 800 may have additional features or functionality. For example, computing device 800 may also include additional data storage devices (removable and/or non-removable) such as, for example, magnetic disks, optical disks, or tape. Such additional storage is illustrated in FIG. 8 by a removable storage 809 and a non-removable storage 810. Computer storage media may include volatile and nonvolatile, removable and non-removable media implemented in any method or technology for storage of information, such as computer-readable instructions, data structures, program modules, or other data. System memory 804, removable storage 809, and non-removable storage 810 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 800. Any such computer storage media may be part of device 800. Computing device 800 may also have input device(s) 812 such as a keyboard, a mouse, a pen, a sound input device, a touch input device, a location sensor, a camera, a biometric sensor, etc. Output device(s) 814 such as a display, speakers, a printer, etc. may also be included. The aforementioned devices are examples and others may be used.

Computing device 800 may also contain a communication connection 816 that may allow device 800 to communicate with other computing devices 818, such as over a network in a distributed computing environment, for example, an intranet or the Internet. Communication connection 816 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 804, including operating system 805. While executing on processing unit 802, programming modules 806 (e.g., application 820 such as a media player) may perform processes including, for example, one or more stages of methods, algorithms, systems, applications, servers, databases as described above. The aforementioned process is an example, and processing unit 802 may perform other processes. Other programming modules that may be used in accordance with embodiments of the present disclosure may include machine learning application.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

Although the invention has been explained in relation to its preferred embodiment, it is to be understood that many other possible modifications and variations can be made without departing from the spirit and scope of the invention as hereinafter claimed.

1-14. (canceled)

15. An electronic wearable patch, comprising:
a flexible energy-storage module including
a first substrate layer, a negative connection layer, a cathode layer, an electrolytic separation layer, an anode layer, a positive connection layer, and a second substrate layer being, and
a sealing adhesive, the first substrate layer and the second substrate layer being peripherally fixed to each other by the sealing adhesive.

16. The electronic wearable patch of claim 15, further comprising:
a controller;
at least one sensing module;
a wireless communication module;
a flexible adhesive; and
a printed circuit board (PCB) having a first board face and a second board face, the controller, the at least one sensing module, the wireless communication module, and the flexible energy-storage module being mounted onto the first board face,
wherein the controller, the at least one sensing module, and the wireless communication module being electronically connected to each other by the PCB,
wherein the controller, the at least one sensing module, and the wireless communication module being electrically connected to the energy-storage module, and
wherein the flexible adhesive is mounted across the second board face.

17. The electronic wearable patch of claim 16, further comprising:
a supercapacitor mounted onto the first board face,
wherein the controller, the at least one sensing module, and the wireless communication module are electrically connected to the energy-storage module through the supercapacitor.

18. The electronic wearable patch of claim 15, wherein the negative connection layer and the positive connection layer each comprise a base portion and a tab portion, the tab portion being peripherally positioned to the base portion, and wherein the tab portion of the negative connection layer extends through the sealing adhesive, and the tab portion of the positive connection layer extends through the sealing adhesive.

19. The electronic wearable patch of claim 15, wherein a combined thickness of the first substrate layer and the negative connection layer is less than the thickness of the cathode layer, and wherein a combined thickness of the second substrate layer and the positive connection layer is less than the thickness of the anode layer.

20. The electronic wearable patch of claim 15, wherein the cathode layer and the anode layer are printed or deposited coatings.

21. The electronic wearable patch of claim 15, wherein the cathode layer and the anode layer comprise a radiation curable polymer.

22. The electronic wearable patch of claim 21, wherein radiation curable polymer comprises an ultraviolet (UV) curable polymer.

23. The electronic wearable patch of claim 21, wherein radiation curable polymer comprises an electron beam radiation curable primer.

24. The electronic wearable patch of claim 21, wherein the radiation curable polymer comprises an oligomer, a dispersant, a self-level additive, or a conductive additive, or combinations thereof.

25. The electronic wearable patch of claim 15, wherein the electrolytic separation layer comprises a radiation curable structure.

26. The electronic wearable patch of claim 25, wherein the radiation curable structure comprises a dissolved salt, functional material, liquid electrolyte or a salt additive, or combinations thereof.

27. The electronic wearable patch of claim 15, wherein the electrolytic separation layer comprises a liquid, gel or solid electrolyte.

28. The electronic wearable patch of claim 15, further comprising a drug reservoir.

29. The electronic wearable patch of claim 28, wherein the drug reservoir comprises a delivery mechanism, the drug reservoir being mounted onto the first board face, and wherein the delivery mechanism is electronically connected with the controller.

30. An electronic wearable patch, comprising:
a flexible energy-storage module including a first substrate layer, a negative connection layer, a cathode layer, an electrolytic separation layer, an anode layer, a positive connection layer, and a second substrate layer, and a sealing adhesive, the first substrate layer and the second substrate layer being peripherally fixed to each other by the sealing adhesive;
a flexible printed circuit board (PCB) having a first board face and a second board face; and
a controller mounted on the PCB first board face and electrically connected to the flexible energy-storage module.

31. The electronic wearable patch of claim 30, further comprising:
at least one sensing module mounted on the flexible PCB and electronically connected to the controller by the PCB; and
a wireless communication module mounted on the first board face and electronically connected to the controller and the at least one sensing module by the PCB,
wherein the at least one sensing module and the wireless communication module are electrically connected to the flexible energy-storage module.

32. The electronic wearable patch of claim 30, wherein
the negative connection layer and the positive connection layer each comprise a base portion and a tab portion, the tab portion being peripherally positioned to the base portion,
the sealing adhesive being positioned around the base portion of the negative connection layer, the cathode layer, the electrolytic separation layer, the anode layer, and the base portion of the negative connection layer,
the tab portion of the negative connection layer traversing through the sealing adhesive, and
the tab portion of the positive connection layer traversing through the sealing adhesive.

33. The electronic wearable patch of claim 32, wherein the cathode layer and the anode layer are radiation curable polymers.

34. The electronic wearable patch of claim 33, wherein radiation curable polymers are cured by ultraviolet (UV) radiation or electron beam radiation.
